(19) United States
(12) Patent Application Publication
(10) Pub. No.: US 2025/0223551 A1
(43) Pub. Date: Jul. 10, 2025

(54) CELLULAR MICROCOMPARTMENTS COMPRISING LYMPHOCYTES FORMING A GROUPED 3D CULTURE AND HAVING A LOW TNF ALPHA CONTENT

(71) Applicant: TREEFROG THERAPEUTICS, PESSAC (FR)

(72) Inventors: Maxime FEYEUX, TALENCE (FR); Camille FIALIN, BORDEAUX (FR)

(21) Appl. No.: 18/853,479

(22) PCT Filed: Apr. 5, 2023
(86) PCT No.: PCT/EP2023/059027

§ 371 Date: Oct. 29, 2024

(30) Foreign Application Priority Data
Apr. 5, 2022 (FR) FR2203095
Apr. 5, 2022 (FR) FR2203100
Dec. 20, 2022 (FR) FR2213989
Dec. 20, 2022 (FR) FR2213998

(51) Int. Cl. C12N 5/078

(52) U.S. Cl. CPC C12N 5/0634; C12N 2501/2302; C12N 2501/2307; C12N 2501/2312; C12N 2501/2315; C12N 2501/2318; C12N 2501/2321; C12N 2501/25; C12N 2513/00; C12N 2533/74

(57) ABSTRACT

The invention relates to a three-dimensional cellular microcompartment or an assembly of three-dimensional cellular microcompartments of ovoid, cylindrical, spheroid or spherical shape or of substantially ovoid, cylindrical, spheroid or spherical shape, the smallest dimension of which is between 200 and 400 μm, comprising an outer hydrogel layer that delimits an internal part, said internal part comprising, in a medium, between 500 and 5000 lymphocytes forming a grouped three-dimensional culture and having a TNF alpha content of less than 100 ng/ml of medium. The invention also relates to a method for producing such a microcompartment or microcompartment assembly.

### TECHNICAL FIELD

The present invention relates to the field of large-scale lymphocyte cell culture. The invention relates in particular to a particular cellular microcompartment, its preparation method and its uses

### PRIOR ART

The progress made over the last few decades has highlighted the benefits of cell therapy in treating a wide range of pathologies.

Cell therapy is defined as the administration of living cells to a patient with the aim of preventing, treating or alleviating a pathology. The aim is to achieve a lasting effect by injecting therapeutic cells.

Numerous cell therapy approaches can be envisaged, such as:
the autologous approach, in which the patient is their own donor.
the allogeneic approach, in which the donor is different from the patient.

Cell therapy covers a wide range of therapeutic fields and applies to numerous cell types, from pluripotent cells to specialized adult cells with metabolic functions (e.g. hepatocytes) or mechanical functions (e.g. myoblasts, chondrocytes), as well as hematopoietic lineages such as mononuclear blood cells (e.g. T lymphocytes or NK lymphocytes).

One of the most promising therapies to date is CAR (chimeric antigen receptor) cell therapy. CAR-T cells are T lymphocytes expressing chimeric antigen receptors, which are artificial receptors designed to recognize an antigen present on the surface of tumor cells. CAR-T cells guide the immune system to target and eradicate cancer cells. This approach is considered a major advance in immunotherapy, and many CAR-T cells are currently being developed to extend their uses. CAR-T cells are generally produced using the autologous approach. However, it is also possible to create allogeneic CAR-T cells.

There is also an alternative using NK (natural killer) lymphocytes. NK lymphocytes are highly potent cytotoxic effector cells with a proven anti-tumor effect.

In order to develop the potential of cell-based therapies using lymphocytes, in particular CAR-T or CAR-NK therapies, it is necessary to be able to rely on large-scale lymphocyte cell culture techniques.

The ex vivo production of immune cells such as T or NK lymphocytes is particularly complex, especially on a large scale. Indeed, the in vitro culture methods developed to date are too stressful and rapidly lead to cell exhaustion, making it impossible to produce lymphocytes on a large scale. In vivo, immune cell proliferation occurs mainly in mechanically stable environments such as solid tissue. As the bioreactor environment is filled with non-physiological shear stresses, the expansion rates obtained are generally still insufficient compared with in vivo expansion capacities.

At present, there is no satisfactory solution for the rapid large-scale production of functional lymphocytes, in particular CAR-T or CAR-NK cells.

Several culture methods are mainly used for CAR cell expansion: static flask culture, static gas-permeable bag culture and rocking motion bioreactor culture (Vormittag P, Gunn R, Ghorashian S, Veraitch F S. Curr Opin Biotechnol. 2018), the G-rex® platform (Ludwig et al. “Methods and Process Optimization for Large-Scale CAR T Expansion Using the G-Rex Cell Culture Platform” Methods Mol Biol 2020; 2086:165-177).

However, these culture methods are not suitable for large-scale lymphocyte production due to exposure of the cells to numerous handling steps that increase the risk of contamination, exposure of the cells to excessive stress, and/or constraints on the development and capacity of the platforms and methods used. Moreover, the work involved in culture and storage would be too demanding for large-scale production.

To remedy this problem, the prior art proposes to activate lymphocytes before they are cultured, in order to achieve rapid lymphocyte proliferation.

Nevertheless, early lymphocyte activation leads to a physiological process of cell exhaustion. Depletion corresponds to the early maturation of naive T cells into a subpopulation of effector T cells, limiting graft persistence in vivo.

Another solution proposed by the prior art is the use of hydrogel and alginate microtubes suspended in a cell culture medium. This method reports a lymphocyte expansion rate of 320 at 14 days of culture (Lin et al. Adv. Healthcare Mater. 2018, 1701297)

However, although this method provides a satisfactory expansion rate, it is not suitable for large-scale culture. This method would require the development of a dedicated culture system and would be too complicated to adapt at large scale.

On the other hand, this method does not guarantee the functional phenotype of the lymphocytes, and consequently that the action of the lymphocytes is long-lasting.

The complexity of the differentiation and function of each lymphocyte subpopulation means that analysis of the phenotype during proliferation is essential to the success of any lymphocyte-based therapy, particularly those using CAR-T or CAR-NK cells.

Less-differentiated T-cell subpopulations such as naive T lymphocytes and Tscm (stem cell-like memory T cell) lymphocytes appear to be particularly important in ensuring a lasting effect in patients (Vita Golubovskaya and Lijun Wu (Cancers 2016, 8, 36). What's more, memory-like NK subpopulations enable persistence of clinical response (Romee et al. “Cytokine activation induces human memory-like NK cells” Blood 2012 Dec. 6; 120(24):4751-60).

On the other hand, we also know that the effector T lymphocyte subpopulation is essential to guarantee a significant effect on the target cell(s).

It therefore appears necessary to control cell sub-populations during large-scale lymphocyte culture to obtain an optimal ratio between persistence and in vivo efficacy.

Current production processes involving lymphocytes are very costly and difficult to scale up, resulting in lymphocytes of mixed quality and reduced persistence.

There is therefore a strong need for a solution enabling large-scale production of lymphocytes with a functional phenotype, to meet a much-needed demand for lymphocyte-based cell therapy.

The aim of the invention is therefore to meet all of these needs and to overcome the disadvantages and limits of the prior art.

### SUMMARY OF THE INVENTION

To meet this objective, the invention proposes to use a particular microcompartment to obtain large-scale production of lymphocytes, suitable for use in cell therapy.

To this end, the object of the invention is a closed three-dimensional cellular microcompartment of ovoid, cylindrical, spheroid or spherical shape or of substantially ovoid, cylindrical, spheroid or spherical shape, the smallest dimension of which is between 200 and 400 μm, comprising an outer hydrogel layer that delimits an internal part, said internal part comprising, in a medium, between 500 and 5000 lymphocytes forming a grouped three-dimensional culture and having an endogenous Tumor Necrosis Factor (TNF alpha) content of less than 100 ng/ml of the medium.

Preferably, the internal part of the microcompartment comprises a perforin content of less than 0.5 μg/mL of the medium and/or a granzyme B content of less than 1 μg/mL of the medium and/or an IL6 content of less than 0.5 μg/ml of the medium and/or a GM-CSF content of less than 0.5 μg/mL of the medium.

Advantageously, the microcompartment according to the invention enables the production of lymphocytes with a functional phenotype while guaranteeing rapid large-scale production.

According to a particularly suitable embodiment, the microcompartment according to the invention comprises lymphocytes forming a grouped three-dimensional, lumen-free culture.

The microcompartment according to the invention can be obtained after encapsulation of lymphocytes with or without addition of extracellular matrix, and according to a variant with or without addition of extracellular matrix element.

Advantageously, the absence of extracellular matrix or matrix element addition at the time of lymphocyte encapsulation improves their expansion rate after 3 days of culture.

According to a preferred object of the invention, the microcompartment according to the invention has been obtained after encapsulation of previously activated lymphocytes. Lymphocyte activation advantageously stimulates lymphocyte proliferation in the internal part of the microcompartment according to the invention. According to one variant, lymphocyte activation can be carried out in the microcompartment after encapsulation.

According to another particularly suitable embodiment, the microcompartment according to the invention comprises lymphocytes expressing a chimeric antigen receptor. When the microcompartment according to the invention comprises T lymphocytes, it preferably comprises a concentration of naive and/or Tscm lymphocyte greater than 20%, even more preferably greater than 40%.

Advantageously, such a high concentration of naive and/or Tscm lymphocytes guarantees a long-lasting effect and avoids the phenomenon of early lymphocyte exhaustion.

According to a preferred object of the invention, the invention concerns a set of microcompartments.

According to another aspect, the invention concerns a process for preparing a microcompartment or a set of microcompartments comprising at least the implementation of the following steps, an incubation, an encapsulation and a culture step. Finally, the invention targets a microcompartment according to the invention for use as a medicament, preferably in the prevention or treatment of autoimmune diseases, immunodeficiency syndromes, cancers, viral diseases or inflammatory diseases.

Other features and advantages will emerge from the detailed description of the invention and the following examples.

### BRIEF DESCRIPTION OF THE FIGURES

FIG. 1a is a phase contrast microscopy image at ×20 magnification of a set of microcompartments according to the invention comprising T lymphocytes after 3 days of encapsulation culture.

FIG. 1b is a phase contrast microscopy image at ×10 magnification of a set of microcompartments according to the invention comprising T lymphocytes after 3 days of encapsulation culture.

FIG. 2 is a representation of the results of tests on the amplification of lymphocytes in microcompartments according to the invention.

FIG. 3 shows the expansion factor of T lymphocytes after 6 days of culture with or without encapsulation.

FIG. 4 is a comparative representation of the cytokine expression profile during T lymphocyte culture after 6 days with or without encapsulation. Results are presented with values normalized to GM-CSF and IFN-γ secretion.

FIG. 5 is a confocal microscopy image of a microcompartment with polarized lymphocytes.

FIG. 6 shows the expansion factor of T lymphocytes after 14 days of culture with encapsulation (“Capsules”) or without encapsulation (“Control”).

FIG. 7a is a comparative representation of cytokine expression during NK lymphocyte culture after 14 days of culture with or without encapsulation (for an A-supernatant donor diluted 10-fold). Results are presented with values normalized to IFN-γ secretion.

FIG. 7b is a comparative representation of granzyme B expression during NK lymphocyte culture after 14 days of culture with or without encapsulation (for an A-supernatant donor diluted 100-fold). Results are presented with values normalized to IFN-γ secretion.

FIG. 8a is a comparative representation of cytokine expression during NK lymphocyte culture after 11 days of culture with or without encapsulation (for a B-supernatant donor diluted 10-fold). Results are presented with values normalized to IFN-γ secretion.

FIG. 8b is a comparative representation of granzyme B expression during NK lymphocyte culture after 11 days of culture with or without encapsulation (for a B-supernatant donor diluted 100-fold). Results are presented with values normalized to IFN-γ secretion.

FIG. 9 shows the comparative results of a toxicity test carried out on NK obtained without encapsulation and on NK obtained according to the invention (for donor B).

FIG. 10 shows the comparative results of viability evolution during culture of NK without encapsulation and NK encapsulated according to the invention (results for two donors A and B).

FIG. 11a is a phase contrast microscopy image at ×4 magnification of a set of microcompartments according to the invention containing NKs on the day of encapsulation is shown in FIG. 11a.

FIG. 11b is a phase contrast microscopy image at ×10 magnification of a set of microcompartments according to the invention containing NK after 11 days of encapsulation culture.

FIG. 12a shows the expansion factor of NK lymphocytes when cultured for 12 days with encapsulation (“Capsules” encapsulation without activation bead) or without encapsulation (“Control”).

FIG. 12b shows comparative results for the evolution of viability when culturing NKs without encapsulation (“control”) and NKs encapsulated according to the invention (“capsule” encapsulation without activation beads) (results for two donors B and C).

### DETAILED DESCRIPTION OF THE INVENTION

### Definitions

For the purposes of the invention, “alginate” means linear polysaccharides formed from β-D-mannuronate and a-L-guluronate, salts and derivatives thereof.

For the purposes of the invention, “hydrogel capsule” or “hydrogel microcompartment” means a three-dimensional structure formed from a matrix of polymer chains, swollen using a liquid, preferentially water.

For the purposes of the invention, “human cells” means human cells or immunologically humanized non-human mammalian cells. Even when this is not specified, the cells, stem cells, progenitor cells and tissues according to the invention consist of or are obtained from human cells or from immunologically humanized non-human mammalian cells.

For the purposes of the invention, the term “mutant cell” refers to a cell carrying at least one mutation.

“Feret diameter” of a microcompartment (or of a part of a microcompartment) according to the invention means the distance “d” between two tangents to said microcompartment (or to said part), these two tangents being parallel, such that the entire projection of said microcompartment (or of said part) is between these two parallel tangents. A Feret diameter of the internal part of the microcompartment is measured between two interfaces of the internal part and the external layer of the microcompartment, i.e. the distance “d” between two tangents to said internal part, these two tangents being parallel, such that the entire projection of said internal part is between these two parallel tangents.

For the purposes of the invention, “variable thickness” of a layer means that, for a single microcompartment, the layer does not have the same thickness throughout.

For the purposes of the invention, “microcompartment” or “capsule” means a partially or entirely closed three-dimensional structure containing several cells.

Within the meaning of the invention, “medium” means an aqueous solution including cells, compatible with the survival, development and/or metabolism of the cells. It may be a culture medium.

For the purposes of the invention, “convective culture medium” means a culture medium stirred by internal movements.

For the purposes of the invention, the term “mutation” means a genetic or epigenetic mutation, preferentially a functional mutation. It may in particular involve a point modification of the genetic sequence, a structural variant, an epigenetic modification, or a modification of the mitochondrial DNA.

The term “functional mutation” within the meaning of the invention refers to a transmissible genetic or epigenetic modification which confers a potential gain or loss of function or loss of potential function to a relevant mutant cell. It preferably involves a mutation causing a modification of the phenotype of the affected mutant cell. Very preferentially, it involves a change of the genomic and/or of the epigenomic sequence which alters the therapeutic potential of a population of cells, or by increasing the risk associated with the therapy produced, or by decreasing the benefit provided by the therapy produced.

For the purposes of the invention, “smallest dimension” of a microcompartment or of a cell layer means the value of the smallest Feret diameter of said microcompartment. For the purposes of the invention, “lumen” means a volume of aqueous solution topologically surrounded by cells. Preferentially, its content is not in diffusive equilibrium with the volume of convective liquid present outside the microcompartment.

“Smallest radius” of the internal part of a microcompartment according to the invention means half of the value of the smallest Feret diameter of the internal part of the microcompartment.

“Average radius” of the internal part of a microcompartment according to the invention means the average of the radii of the smallest compartment, each radius corresponding to half the value of a Feret diameter of the internal part of the microcompartment.

“Expansion rate at X days” according to the invention means a measurement of cell proliferation at time t=X. The rate of expansion is measured by taking the ratio of the number of cells counted on day X of the culture divided by the number of cells at the start of the culture (day of encapsulation or day of placement in culture).

According to the invention, “large-scale culture” means a cell culture method suitable for a lymphocyte production batch of liver microtissue making it possible to treat at least 1 patient, preferably 10 patients, more preferably 100 patients, even more preferably more than 1,000 patients.

“Functional phenotype” according to the invention means lymphocytes with a conventional membrane marker expression profile (CD3+, CD4+ or CD8+ for T lymphocytes, and CD3−, CD56+ for NK lymphocytes), known to the person skilled in the art, and a capacity to secrete interleukins (IL2, IL6, IL10, IL12) or other factors of interest (granzyme B, INF gamma, GMCSF, and TNF alpha) under induction of antigen presentation in vitro.

For the purposes of the invention, “progenitor cell” means a stem cell that is already engaged in lymphocyte cell differentiation but that has not yet differentiated.

For the purposes of the invention, “embryonic stem cell” means a pluripotent stem cell of cells derived from the internal cell mass of the blastocyst. The pluripotency of the embryonic stem cells can be evaluated by the presence of markers such as the transcription factors OCT4, NANOG and SOX2 and surface markers such as SSEA3/4, Tra-1-60 and Tra-1-81. The embryonic stem cells used in the context of the invention are obtained without destroying the embryo from which they originate, for example using the technique described in Chang et al. (Cell Stem Cell, 2008, 2(2): 113-117).

Optionally, embryonic stem cells from humans can be excluded.

For the purposes of the invention, “pluripotent stem cell” or “pluripotent cell” means a cell which has the capacity to form all the tissues present in the entire organism of origin, without however being able to form an entire organism per se. Human pluripotent stem cells can be called hPSC in the present application. These may in particular be induced pluripotent stem cells (iPSC or hiPSC for human induced pluripotent stem cells), embryonic stem cells or MUSE cells (for “multilineage-differentiating stress enduring”).

For the purposes of the invention, “induced pluripotent stem cell” means a pluripotent stem cell induced to become pluripotent by genetic reprogramming of differentiated somatic cells. These cells are in particular positive for pluripotency markers, such as staining with alkaline phosphatase and expression of the proteins NANOG, SOX2, OCT4 and SSEA3/4. Examples of methods for obtaining induced pluripotent stem cells are described in the articles by Yu et al. (Science 2007, 318 (5858): 1917-1920), Takahashi et al (Cell, 207, 131(5): 861-872) and Nakagawa et al. (Nat Biotechnol, 2008, 26(1): 101-106).

### Cellular Microcompartment

An object of the invention is therefore a closed three-dimensional cellular microcompartment of ovoid, cylindrical, spheroid or spherical shape or of substantially ovoid, cylindrical, spheroid or spherical shape, the smallest dimension of which is between 200 and 400 μm, comprising an outer hydrogel layer that delimits an internal part, said internal part comprising, in a medium, between 500 and 5000 lymphocytes forming a grouped three-dimensional culture and having a TNF alpha content of less than 100 ng/ml of the medium.

Preferably, the internal part of the microcompartment comprises a TNF alpha content of less than 50 ng/ml of medium, in particular less than 30 ng/ml, more preferably less than 10 ng/ml, even more preferably less than 3 ng/ml. The microcompartment thus comprises at least lymphocytes that secrete little or no TNF alpha.

In the context of the invention, the content of molecule(s) (in particular cytokines such as TNF, granzyme B, perforin, etc.) in the medium internal to the microcompartment can be measured directly in the capsule, or in the medium external to the microcompartment when the latter is in a medium. Indeed, molecules smaller than 150 KDa are in osmotic equilibrium between the internal part of the microcompartment and the medium in which the microcompartment is located.

According to a preferred embodiment, the internal part of the microcompartment comprises a perforin content of less than 0.5 μg/ml of medium, preferably less than 0.1 μg/mL, more preferably less than 50 ng/ml, even more preferably less than 10 ng/ml.

A further object of the invention is a closed three-dimensional cellular microcompartment of ovoid, cylindrical, spheroid or spherical shape or of substantially ovoid, cylindrical, spheroid or spherical shape, the smallest dimension of which is between 200 and 400 μm, comprising an outer hydrogel layer that delimits an internal part, said internal part comprising, in a medium, between 500 and 5000 lymphocytes forming a grouped three-dimensional culture and having a perforin content of less than 0.5 μg/ml of medium, preferably less than 0.1 μg/mL, more preferably less than 50 ng/ml, even more preferably less than 10 ng/ml.

According to a preferred embodiment, the internal part of the microcompartment comprises a granzyme B content of less than 1 μg/ml of medium, in particular less than 0.5 μg/mL of medium, more preferably less than 0.1 μg/mL, even more preferably less than 50 ng/ml. The microcompartment thus comprises at least lymphocytes that secrete little or no granzyme B.

A further object of the invention is therefore a closed three-dimensional cellular microcompartment of ovoid, cylindrical, spheroid or spherical shape or of substantially ovoid, cylindrical, spheroid or spherical shape, the smallest dimension of which is between 200 and 400 μm, comprising an outer hydrogel layer that delimits an internal part, said internal part comprising, in a medium, between 500 and 5000 lymphocytes forming a grouped three-dimensional culture and having a granzyme B content of less than 1 μg/ml of medium. According to a preferred embodiment, when the lymphocytes are NK lymphocytes, the internal part of the microcompartment comprises an IL6 content of less than 0.5 μg/mL of medium, preferably less than 0.1 μg/mL, more preferably less than 50 ng/mL, even more preferably less than 10 ng/mL.

A further object of the invention is a closed three-dimensional cellular microcompartment of ovoid, cylindrical, spheroid or spherical shape or of substantially ovoid, cylindrical, spheroid or spherical shape, the smallest dimension of which is between 200 and 400 μm, comprising an outer hydrogel layer that delimits an internal part, said internal part comprising, in a medium, between 500 and 5000 NK lymphocytes forming a grouped three-dimensional culture and having an IL6 content of less than 0.5 μg/mL of medium, preferably less than 0.1 μg/mL, more preferably less than 50 ng/ml, even more preferably less than 10 ng/ml.

According to a preferred embodiment, when the lymphocytes are NK lymphocytes, the internal part of the microcompartment comprises a GM-CSF content of less than 0.5 μg/mL of medium, preferably less than 0.1 μg/mL, more preferably less than 50 ng/ml, even more preferably less than 10 ng/mL.

A further object of the invention is a closed three-dimensional cellular microcompartment of ovoid, cylindrical, spheroid or spherical shape or of substantially ovoid, cylindrical, spheroid or spherical shape, the smallest dimension of which is between 200 and 400 μm, comprising an outer hydrogel layer that delimits an internal part, said internal part comprising, in a medium, between 500 and 5000 NK lymphocytes forming a grouped three-dimensional culture and having a GM-CSF content of less than 0.5 μg/mL of medium, preferably less than 0.1 μg/mL, more preferably less than 50 ng/ml, even more preferably less than 10 ng/mL.

According to one embodiment an object of the invention is a closed three-dimensional cellular microcompartment of ovoid, cylindrical, spheroid or spherical shape or of substantially ovoid, cylindrical, spheroid or spherical shape, the smallest dimension of which is between 200 and 400 μm, comprising an outer hydrogel layer that delimits an internal part, said internal part comprising, in a medium, between 500 and 5000 T lymphocytes forming a grouped three-dimensional culture and having a content of less than 100 ng/ml of the medium and:
a perforin content of less than 0.5 μg/mL of medium, and/or
a granzyme B content of less than 1 μg/mL of medium.

According to one embodiment, an object of the invention is a closed three-dimensional cellular microcompartment of ovoid, cylindrical, spheroid or spherical shape or of substantially ovoid, cylindrical, spheroid or spherical shape, the smallest dimension of which is between 200 and 400 μm, comprising an outer hydrogel layer that delimits an internal part, said internal part comprising, in a medium, between 500 and 5000 NK lymphocytes forming a grouped three-dimensional culture and having a TNF-α content of less than 100 ng/ml of the medium and:
a perforin content of less than 0.5 μg/mL of medium, and/or
a granzyme B content of less than 1 μg/mL of medium, and/or
a perforin IL6 content of less than 0.5 μg/mL of medium, and/or
a GM-CSF content below 0.5 μg/mL of medium.

According to the invention, surprisingly, a TNF alpha content of less than 100 ng/ml of medium in the internal part of the microcompartment, in particular less than 30 ng/ml, preferably less than 50 ng/ml, more preferably less than 10 μg/mL, even more preferably less than 3 ng/ml, is a criterion guaranteeing the functional phenotype of lymphocytes within the internal part of the microcompartment according to the invention.

Tumor necrosis factor (TNF alpha) is a pleiotropic cytokine involved in numerous physiological processes that control inflammation, tumor response and immune system homeostasis. TNF alpha can be secreted by lymphocytes or other cells (notably activated macrophages) to act directly on cells infected by certain pathogens or to promote lymphocyte proliferation. TNF alpha can thus promote activation and proliferation of naive and effector T cells, but can also induce apoptosis of activated effector T cells. TNF alpha is stored in intracellular secretory vesicles. These vesicles are typically released into the immediate environment of the target cells.

In conventional culture methods, lymphocytes are subjected to various physical constraints and/or stresses which tend to trigger the release of interleukins, particularly TNF alpha. This uncontrolled release of TNF alpha alters the direct environment of lymphocytes, and can significantly reduce their viability by inducing apoptosis in some of them. On the other hand, the remaining lymphocytes no longer display a functional phenotype. Indeed, lymphocytes have lost their ability to secrete interleukins under the induction of antigen presentation, thus affecting their ability to act on the target antigen.

Advantageously, the microcompartment according to the invention makes it possible to obtain lymphocytes with a functional phenotype.

Contrary to what is suggested in the prior art, a TNF alpha content of less than 100 ng/ml of medium, in particular less than 30 ng/ml, preferably less than 50 ng/ml, more preferably less than 10 μg/mL, even more preferably less than 3 ng/ml is particularly sought-after in lymphocyte culture.

A TNF alpha concentration greater than 100 ng/ml of medium and/or a perforin concentration greater than 0.5 μg/mL and/or a granzyme B concentration greater than 1 μg/ml, would indicate that an uncontrolled process of interleukin release is taking place in the microcompartment according to the invention, making it incompatible with large-scale culture of lymphocytes with a functional phenotype.

The same applies to NK lymphocytes with a concentration above 0.5 μg/mL IL6 and/or a concentration above 0.5 μg/mL GM-CSF. IL6 in particular is known to decrease NK cytotoxic activity (Cifadi et al., “Inhibition of natural killer cell cytotoxicity by interleukin-6: implications for the pathogenesis of macrophage activation syndrome” Artritis Rheumatol, 2015 November; 67(II): 3037-46. doi: 10.1002/art.39295.). A reduction in IL6 concentration therefore has the advantage of increasing NK efficacy.

Advantageously, the microcompartment according to the invention is suitable for large-scale culture.

The microcompartment according to the invention advantageously has a T lymphocyte expansion rate of at least 20-fold after 3 days in culture, and an NK lymphocyte expansion rate of at least 40-fold after 14 days in culture.

The microcompartment according to the invention comprises an external hydrogel layer. Preferentially, the hydrogel used is biocompatible, that is to say it is non-toxic to the cells. The external hydrogel layer must allow the diffusion of oxygen and nutrients in order to supply the cells contained in the microcompartment and to enable them to survive. According to one embodiment, the external hydrogel layer comprises at least alginate. It may consist exclusively of alginate. The alginate can in particular be a sodium alginate, composed of 80% α-L-guluronate and 20% α-D-mannuronate, with an average molecular weight of 100 to 400 kDa and a total concentration of between 0.5 and 5% by weight. The external hydrogel layer has no cells.

The outer hydrogel layer makes it possible in particular to protect the cells from the mechanical stress of bioreactors, to limit the uncontrolled secretion by lymphocytes in a cytokine culture that are potentially toxic when accumulated in the medium.

The average outer layer thickness can be variable. It is preferably between 20 and 60 μm, more preferably between 30 and 40 μm. The ratio between the smallest radius of the internal part and this thickness is preferably between 2 and 10 μm.

The microcompartment according to the invention can be obtained after encapsulation of lymphocytes without addition of extracellular, natural or synthetic matrix.

According to a variant, the microcompartment according to the invention does not comprise, in its internal part, extracellular matrix such as Matrigel® and/or Geltrex® and/or a hydrogel-type matrix of plant origin such as modified alginates or of synthetic origin or a copolymer of poly (N-isopropylacrylamide) and poly (ethylene glycol) (PNIPAAm-PEG) of the Mebiol® type.

According to a variant, the microcompartment according to the invention can be obtained after encapsulation of lymphocytes without addition of extracellular matrix. The extracellular matrix elements can be peptide or peptidomimetic sequences, mixtures of proteins, extracellular compounds or structural proteins, such as collagen, laminins, entactin, vitronectin, as well as growth factors or cytokines.

The absence of extracellular matrix elements in the internal part of the microcompartment according to the invention after encapsulation guarantees lymphocytes freedom of structural organization within the microcompartment according to the invention.

Advantageously, the absence of extracellular matrix elements improves lymphocyte expansion rates.

Within the microcompartment, lymphocytes group together in three dimensions, preferably in clusters.

Preferably, the microcompartment according to the invention comprises lymphocytes forming a grouped three-dimensional lumen-free culture.

The absence of a lumen advantageously limits the autocrine and paracrine signaling of lymphocytes, making their phenotype particularly stable from encapsulation onwards.

Advantageously, the absence of a lumen also makes it easier to control the size of lymphocyte clusters present in the microcompartment.

The microcompartment according to the invention can be obtained after encapsulation of previously activated lymphocytes, thereby increasing their expansion rate by stimulating their proliferation. Activation can be achieved by bringing lymphocytes into contact with artificial antigen presenting cells (aAPCs), most preferably via CD3/CD28 antibodies for T lymphocytes, or via CD2/NKp46 antibodies for NK lymphocytes.

When lymphocytes have been previously activated and to maintain their activation, the internal part of the microcompartment according to the invention can comprise a solution comprising cytokines, notably 1L2 or the combination of IL7 and IL15 for T lymphocytes, notably IL2, IL12, IL18, IL21 (preferably IL2 or IL12 or IL18 or IL21 or the combination of at least two cytokines chosen from IL2, IL12, IL18 and IL21) for NK.

Cytokines in the internal part of the microcompartment accelerate lymphocyte proliferation.

In another embodiment, the microcompartment comprises lymphocytes activated during and/or after encapsulation. In this embodiment, the lymphocytes can be co-encapsulated with artificial antigen-presenting cells, e.g. activation beads coated with suitable antibody(s) (e.g. Cloudz Human NK Cell Expansion Kit (R&D Systems) or NK Cell Activation/Expansion Kit (Miltenyi Biotec)).

Preferably, in this embodiment, lymphocyte activation takes place within the microcompartment. Preferably, lymphocyte activation within the microcompartment is achieved at a lymphocyte/antigen quantity ratio of greater than 8:1, preferably greater than 9:1, even more preferably greater than 10:1.

Advantageously, activation is carried out in the microcompartment by controlling the ratio of the number of lymphocytes to the amount of antigen required, so as to activate the lymphocytes while limiting cell exhaustion.

The invention's microcompartment isolates lymphocytes, preventing cell mixing or convection. This mechanical isolation makes it possible to approximate the spatial and structural organization of lymphocytes in vivo.

Thanks to the mechanical isolation provided by the microcompartment, lymphocyte activation cannot be homogeneous or continuous. In this way, activation approximates the conditions of in vivo lymphocyte activation, favoring their polarization.

Preferably, the lymphocytes forming the clusters are polarized. The polarity of these lymphocytes within each cluster can be demonstrated by the eccentric positioning of the nucleus within the cell. Polarized lymphocytes can also exhibit anisotropies in the distribution of Dig, Scrib and Lgl proteins.

Polarization of lymphocytes in the microcompartment may be an indicator of the functional phenotype. Indeed, if a lymphocyte contains a sufficient quantity of granules and vesicles containing interleukins, notably TNF alpha, then the lymphocytes are polarized.

Unexpectedly, the invention's microcompartment retains the clustered conformation of lymphocytes, enabling better proliferation while maintaining a functional phenotype. Consequently, this makes it possible to reduce the number of passages and to reduce the time in culture necessary to reach the final number of lymphocytes required.

The microcompartment may also comprise, in addition to clusters, cells in suspension in the microcompartment.

The microcompartment according to the invention preferably comprises a concentration of naive and/or Tscm lymphocytes greater than 20%, even more preferably greater than 40%.

The concentration of naive and/or Tscm lymphocytes ensures a lasting effect during cell therapy. It is particularly important during culture to obtain a naive and/or Tscm lymphocyte content of over 20% in order to obtain an optimal ratio between persistence of effect and in vivo efficacy.

A naïve and/or Tscm lymphocyte concentration of less than 20% would be indicative of a too-short lymphocyte lifespan, not guaranteeing a persistent effect.

Naïve and Tscm lymphocytes are immature lymphocytes displaying CD3+ CD45RO− CD45RA+ CCR7+ markers that can be easily identified and quantified by detection methods well known to those skilled in the art, such as flow cytometry.

According to a particular embodiment, the invention relates to a closed three-dimensional cellular microcompartment of ovoid, cylindrical, spheroid or spherical shape or substantially ovoid, cylindrical, spheroid or spherical shape, the smallest dimension of which is between 200 and 400 μm, comprising an outer hydrogel layer that delimits an internal part, said internal part forming in a medium a grouped three-dimensional culture of lymphocytes having:
a TNF alpha content of less than 100 ng/ml of medium, and
a granzyme B content of less than 1 μg/mL of medium; and/or
a perforin content of less than 0.5 μg/mL of medium.

In this embodiment, when the lymphocytes are T lymphocytes, the proportion of naive T lymphocytes and/or Tscm lymphocytes remains above 20%.

Preferably, the microcompartment is obtained after encapsulation of lymphocytes without addition of extracellular matrix and/or without addition of extracellular matrix elements.

The lymphocytes present in the microcompartment according to the invention were preferably obtained after at least two cell division cycles after encapsulation, in an external hydrogel layer, of at least one lymphocyte.

Preferably, the lymphocytes present in the microcompartment according to the invention have been obtained after at least 2, 3, 4, 5, 6, 7, 8, 9, 10, 11, 12, 13, 14, 15, 20, 25, 28, 30 cell division cycles after encapsulation in an outer hydrogel layer, preferably at least 5, even more preferably at least 6, to particularly obtain between 5 and 100 lymphocytes per lymphocyte encapsulated. For example, the lymphocytes present in the microcompartment were obtained after at least six cell division cycles after the encapsulation of the cells in the external hydrogel layer.

Preferentially, the number of cell divisions for the implementation of the method according to the invention is less than 100, even more preferentially less than 30.

Preferably, the microcompartment is obtained after at least 2 passes after encapsulation, more preferably at least 3, 4 or 5 passes. Each pass can for example last between 2 and 10 days, in particular between 2 and 4 days.

Preferably, the microcompartment is obtained after at least one re-encapsulation, more preferably between 1 and 14 re-encapsulations, in particular between 2 and 7 re-encapsulations. Very preferentially, a re-encapsulation corresponds to a new pass and each encapsulation cycle corresponds to a pass.

Preferably, the microcompartment according to the invention was obtained in less than 6 days after encapsulation, even more preferably in less than 4 days after encapsulation of at least 5 lymphocytes in the internal part delimited by the outer hydrogel layer.

The microcompartment according to the invention can contain between 500 and 5000 lymphocytes, preferably between 1000 and 3000 lymphocytes.

Advantageously, the microcompartment according to the invention protects the lymphocytes from mechanical stress, thus making it possible to obtain a rate of expansion that is particularly suited to large-scale culture.

The microcompartment according to the invention can be in any three-dimensional form, that is, it may have the shape of any object in space. The microcompartment may have any form compatible with cell encapsulation. Preferentially, the microcompartment according to the invention is in a spherical or elongated or substantially spherical or elongated shape. It may have the shape of an ovoid, a cylinder, a spheroid or a sphere or substantially this shape.

It is the outer layer of the microcompartment, that is the hydrogel layer, which imparts its size and shape to the microcompartment according to the invention. Preferentially, the smallest dimension of the microcompartment according to the invention is between 200 μm and 400 μm, preferentially between 200 μm and 350 μm, even more preferentially 200 μm and 300 μm, in particular between 200 μm and 250 μm.

Its largest dimension is preferentially greater than 10 μm, more preferentially between 10 μm and 1 m, even more preferentially between 202 μm and 50 cm.

According to a particularly preferred embodiment, the lymphocytes encapsulated in the microcompartment according to the invention are T lymphocytes or NK lymphocytes.

Preferably, the lymphocytes included in the microcompartment according to the invention express a chimeric antigen receptor.

The microcompartment according to the invention is thus particularly suitable for large-scale lymphocyte cultures for CAR-T or CAR-NK cell therapy.

According to one embodiment, the microcompartment according to the invention does not comprise human embryonic stem cells and/or is not obtained from human embryonic stem cells.

The microcompartment according to the invention may optionally be frozen to be stored. It should then preferentially be thawed before it is used.

The invention also relates to a plurality of microcompartments together.

The invention also relates to an assembly or series of microcompartments comprising at least two three-dimensional cellular microcompartments, characterized in that at least one microcompartment is a microcompartment according to the invention.

Preferably, the series of microcompartments according to the invention is in a culture medium, in particular in an at least partially convective culture medium. Any culture medium suitable for lymphocyte culture can be used, such as, for example, “TexMACS (Miltenyi Biotec)” or “VIVO 15-20 (Lonza)” or “CTS™ OpTmizer™ (ThermoFisher)” or NKMACS (Miltenyi Biotec) or ExCellerate Human NK Cell Expansion Media (R&D Systems), provided that the concentration of dissolved salts is compatible with the maintenance of alginate cross-linking by divalent cations.

According to a particularly suitable embodiment, the object of the invention is a series of cellular microcompartments as described above in a closed chamber, such as a bioreactor, preferentially in a culture medium in a closed chamber, such as a bioreactor. Thus, preferentially, the microcompartments are arranged in a culture medium in a closed bioreactor.

The assembly or series of microcompartments according to the invention preferentially comprises between 2 and 10¹⁶ microcompartments.

The microcompartments according to the invention may be used for all applications, in particular as a medication in cell therapy in humans or animals.

Preferably, the microcompartments according to the invention can be used in the prevention or treatment of autoimmune diseases, immunodeficiency syndromes, cancers, viral diseases or inflammatory diseases.

Thus, the microcompartment according to the invention is suitable for large-scale culture, providing lymphocytes with a functional phenotype guaranteeing optimal efficacy and persistence in vivo.

### Method for Preparing a Microcompartment According to the Invention

The invention also relates to a method for preparing microcompartments according to the invention.

The microcompartment according to the invention can be obtained from lymphocytes or cells capable of differentiating into lymphocytes (stem cells, in particular induced pluripotent stem cells, hematopoietic stem cells including CD34+ cells, etc.).

In a first embodiment, the microcompartment according to the invention is obtained from lymphocytes.

The method for preparing a microcompartment or an assembly of microcompartments according to the invention may comprise the following steps:
(a) incubating lymphocytes in a culture medium comprising cytokines
(b) encapsulating lymphocytes washed in a culture medium in a hydrogel layer, preferably without adding extracellular matrix or without adding an extracellular matrix element, to form a closed three-dimensional microcompartment of ovoid, cylindrical, spheroid or spherical shape or substantially ovoid, cylindrical, spheroid or spherical shape, the smallest dimension of which is between 200 and 400 μm, so that each microcompartment comprises at least 5 lymphocytes in its internal part.
(c) culturing the microcompartments obtained in step (b) in a culture medium containing cytokines, for 2 to 6 days.

The incubation step (a) of the method according to the invention comprises between 0.5 and 2 million lymphocytes per mL of culture medium, preferably 1 million lymphocytes per mL of culture medium.

The lymphocytes in step (a) are preferably T lymphocytes and/or NK lymphocytes.

Preferably, the preparation method according to the invention comprises a step prior to lymphocyte incubation, which consists in activating said lymphocytes using an artificial activation system (aAPC) for 3 days prior to encapsulation.

In a particular embodiment, lymphocyte activation is performed using an artificial activation system (aAPC) within the microcompartment resulting from step (b). Any culture medium suitable for culturing lymphocytes, particularly T lymphocytes or NK lymphocytes, can be used, such as “TexMACS (Miltenyi Biotec)” or “VIVO 15-20 (Lonza)” or “CTS™ OpTmizer™ (ThermoFisher)” medium, for example. Each microcompartment obtained in step (b) comprises at least 0.5 million cells per mL of volume of the internal part of the microcompartment, more preferably at least 1 million cells/mL, in particular at least 2 million cells/mL, even more preferably 5 million cells per mL.

Preferably, each microcompartment obtained in step (b) comprises at least 5 lymphocytes, preferably at least 8 lymphocytes in its internal part.

The cytokines contained in the culture medium may be, for example, one or more cytokines such as IL-2 or a mixture of IL-7 and IL-15, or IL-17 and/or IL-18 and/or IL-21 or other cytokines known to the skilled person to stimulate lymphocyte proliferation.

Preferably, the culture media used in the preparation method of the invention for T lymphocytes are supplemented with 100 to 1000 units/mL IL2 or the combination of 300 to 600 units/mL IL7 and 50 to 100 units/mL IL15.

In the method according to the invention, all of the lymphocytes initially encapsulated in step (b) preferentially represents a volume less than 50% of the volume of the microcompartment in which they are encapsulated, more preferentially less than 40%, 30%, 20%, 10% of the volume of the microcompartment in which they are encapsulated.

The encapsulated lymphocytes are suspended in the form of single cells and/or cluster(s) or assembly(-ies) of at least two cells (“cluster(s)”/groups). Preferably, when there are at least 15 lymphocytes, the single cell(s) represent less than 50% by number of all the cells initially encapsulated in step (b).

Preferably, each lymphocyte cluster initially encapsulated in step (b) has a larger dimension less than 20% of the largest dimension of a microcompartment in which it is encapsulated, even more preferentially less than 10%. Indeed, the lymphocyte groups must not have a size that is too large compared to the size of the microcompartment, since a dimension of these initial cell clusters that is too large could lead, during cell divisions, to premature cell confluence in the capsule; this premature confluence of all or part of the capsules could lead to an increase in intracellular pressures and lead to cellular stress, affecting cellular growth, but also chromosome segregation.

The encapsulation step (b) is implemented according to techniques known to a person skilled in the art. Indeed, any method of producing cell microcompartments containing inside a hydrogel capsule and cells can be used to implement the preparation method according to the invention. In particular, it is possible to prepare microcompartments by adapting the method and the microfluidic device described in Alessandri et al., 2016 (“A 3D printed microfluidic device for production of functionalized hydrogel microcapsules for culture and differentiation of human Neuronal Stem Cells (hNSC)”, Lab on a Chip, 2016, vol. 16, no. 9, pp. 1593-1604), in accordance with the steps described below.

Preferably step (b) is carried out without adding extracellular matrix, either natural or synthetic. According to one variant, step (b) is carried out without adding extracellular matrix elements.

Preferentially, step (b) is implemented in a device capable of generating hydrogel capsules using a microfluidic chip. For example, the device may comprise syringe pumps for several solutions injected concentrically by virtue of a microfluidic injector, which makes it possible to form a jet which breaks up into droplets which are then collected in a calcium bath. According to a particularly suitable embodiment, two or three solutions loaded on two or three syringe pumps:
a hydrogel solution, for example alginate,
optionally an isotonic intermediate solution, preferentially an isotonic solution containing no divalent cations such as Ca2+Mg2+, to avoid excessively premature crosslinking of the hydrogel in the injector, such as for example a sorbitol solution,
the solution coming from step (a) comprising lymphocytes, culture medium.

The three solutions are co-injected (simultaneously injected) concentrically by virtue of a microfluidic injector or microfluidic chip which makes it possible to form a jet that breaks up into droplets, the external layer of which is the hydrogel solution and the core of which is the solution from step (a) comprising lymphocytes; these droplets are collected in a calcium bath which crosslinks and/or gels the alginate solution to form the shell.

To improve the monodispersity of the cellular microcompartments, the hydrogel solution is preferentially charged with a direct current at (between 1 and 10 KV). A ring to ground may optionally be arranged at a distance from the tip of between 1 mm and 20 cm, preferentially 3 mm to 10 cm, even more preferentially 1 cm to 5 cm, from the tip in the plane perpendicular to the axis of the jet exiting the microfluidic injector (coextrusion chip), to generate the electric field.

According to the invention, it is necessary to generate capsules, the internal part of which has an average radius or smaller radius of at least 100 μm. To generate capsules with such dimensions with a coextrusion chip (microfluidic injector or microfluidic chip), the invention in particular proposes modifying the flow rate of the coextruded solutions and the final opening of the co-extrusion chip. “Flow rate” means the flow rate of each solution arriving at the injector. “Final opening of the coextrusion chip” means the internal opening of the outlet channel of the chip.
for the flow rate: the method preferentially goes, for each coextruded solution for the standard capsule sizes known from the prior art, from a value of between 20 and 40 mL/h to a value of between 45 and 150 mL/h, preferentially between 45 and 110 mL per hour for a variant of the invention,
for the final opening diameter of the coextrusion chip (microfluidic injector), which goes from a value preferentially between 50 and 120 μm for the standard capsules sizes known from the prior art, to a diameter value of between 150 and 300 μm, preferentially between 180 and 240 μm.

Thus, according to a particular embodiment, the encapsulation step (c) is carried out using a microfluidic injector, the final opening diameter of which is between 150 and 300 μm, preferentially between 180 and 240 μm, and with the flow rate of each of the 3 solutions of between 45 and 150 mL/h, preferentially between 45 and 110 mL/h.

According to one embodiment, steps (a) and/or (c) are implemented with permanent or sequential stirring. This stirring is important because it maintains the homogeneity of the culture environment and avoids the formation of any diffusion gradient. For example, it allows homogeneous control of cellular oxygenation level; thus avoiding phenomena of hypoxia-related necrosis or hyperoxia-related oxidative stress.

The method according to the invention is preferentially carried out in a closed chamber such as a closed bioreactor.

In a preferred variant, the method according to the invention comprises at least one re-encapsulation of the lymphocytes after step (c), i.e. at least two encapsulation cycles. Preferably, each encapsulation cycle corresponds to a pass. In this variant of the method (at least one re-encapsulation of the cells after step (e)), the number of cell divisions of the entire method (for all passes) is at least 3, 4, 5, 6, 7, 8, 9, 10, 11, 12, 13, 14, 15, 20, 25, 30 cell division cycles.

In a method according to the invention, there may be several re-encapsulations, preferentially between 1 and 100, in particular between 1 and 10 re-encapsulation(s).

According to a particular embodiment, a lymphocyte activation step using an artificial activation system (aAPC) can be carried out within the microcompartment after at least one re-encapsulation.

Each re-encapsulation can comprise:
a step which consists of dissociating the microcompartment or the series of microcompartments in order to obtain a suspension of lymphocytes or a suspension of lymphocyte clusters; the external hydrogel layer may be eliminated in particular by hydrolysis, dissolution, piercing and/or breaking by any biocompatible means, that is to say means which are not toxic for the cells. For example, the elimination may be accomplished using phosphate-buffered saline, a divalent ion chelator, an enzyme such as alginate lyase if the hydrogel comprises alginate, and/or laser microdissection, and
a lymphocyte activation step using an artificial activation system (aAPC), preferably using CD3/CD28 antibodies;
a step of re-encapsulating all or part of the lymphocytes or lymphocyte clusters in a hydrogel capsule. Re-encapsulation is a means suitable for increasing the cell amplification resulting from the pluripotent step, and reducing the risks of mutation.

Re-encapsulation involves removing the outer layer of hydrogel, preferably by resuspending the cells in a partially or totally dissociated manner were in the form of cysts in the micro-compartments and to restart the steps of the method.

According to one embodiment, the re-encapsulation comprises the following steps:
(i) removing the external hydrogel layer,
(ii) resuspending the lymphocytes that were contained in the microcompartment so as to obtain single cells and/or at least one assembly or cluster of lymphocytes in a culture medium containing cytokines
(iii) encapsulating the solution of lymphocytes in a hydrogel layer so as to form a microcompartment of ovoid, cylindrical, spheroid or spherical shape, or substantially ovoid, cylindrical, spheroid or spherical shape, comprising an external hydrogel layer that delimits an internal part, the smallest radius or average radius of said internal part being at least 100 μm;
(iv) culturing the resulting microcompartments in cytokine-containing culture medium,
(vi) preferentially rinsing the microcompartments, so as to eliminate the cytokines;
(vi) culturing the microcompartments in a culture medium without cytokines, for at least one cell division cycle, and
(vii) optionally recovering the resulting cellular microcompartments.

The compartmentalization in microcompartments makes it possible to eliminate the microcompartments containing even more mutated cells than the other capsules. Even if the mutated cells have a rapid growth they will reach the capsular confluence which will limit their multiplication. The compartmentalization also makes it possible not to contaminate the entire cell population, and also to eliminate the capsules containing mutant cells, at any time, in particular before a re-encapsulation step. This sorting may be done either by inline analysis, or by eliminating filled capsules more quickly than others, for example.

In one embodiment, at least one of the steps (preferably all the steps) is carried out at a temperature suited to the survival of the cells, included between 4 and 42° C. The temperature during cell proliferation must preferably be between 32 and 37° C. to avoid triggering mutations by lowering the performance of the repair enzymes. Likewise, preferably, the temperature must be low (ideally about 4° C.) to manage the stress on the cells in step (c).

At any time, the method according to the invention may comprise a step consisting of verifying the phenotype of the cells contained in the microcompartment. This verification can be carried out by identifying the expression by at least some of the cells contained in the microcompartment, of at least 4 genes specific to the desired phenotype chosen from CD45 RA RO, CCR7, CD62L, CD3, CD4, CD8, CD56, CD16.

The cellular microcompartments obtained according to the methods of the invention can then be frozen before any use. The freezing is preferentially carried out at a temperature of between −190° C. and −80° C. The thawing can be carried out by immersing the sealed freezing vessel (screwable ampoule or plastic pouch) in a warm water bath (37 degrees preferentially) so that the cells thaw quite rapidly. The microcompartments according to the invention before they are used may be kept at more than 4° C. for a limited time before they are used, preferentially between 4° C. and 38° C.

Implementing the method according to the invention makes it possible to obtain microcompartments comprising at least 80, preferably at least 500, at least 800, at least 1,000, in particular at least 3,000 lymphocytes.

The invention especially promotes amplification with a high rate of expansion, which consequently reduces the culture time and the number of divisions to obtain a very large number of functional lymphocytes.

According to a second embodiment, the microcompartment according to the invention is obtained from cells capable of differentiating into lymphocytes, and comprises at least the implementation of a method for differentiating cells capable of differentiating into lymphocytes (stem cells, in particular induced pluripotent stem cells, hematopoietic stem cells including CD34+ cells, etc.) into lymphocytes.

Cells capable of differentiating into lymphocytes are preferably selected from:
either stem cells capable of differentiating into lymphocytes, at least into T lymphocytes or NK lymphocytes, preferentially embryonic stem cells or induced pluripotent stem cells or hematopoietic stem cells including CD34+ cells, very preferentially induced pluripotent stem cells, and/or
or progenitor cells capable of differentiating into lymphocytes, at least T lymphocytes or NK lymphocytes,
or differentiated cells capable of undergoing reprogramming so that they become induced pluripotent stem cells capable of differentiating into lymphocytes, at least into T lymphocytes or NK lymphocytes.

In this variant, the method may comprise at least the implementation of the steps of:
producing a cellular microcompartment comprising, inside a hydrogel capsule:
preferably at least extracellular matrix elements or a natural or synthetic extracellular matrix,
cells capable of differentiating into lymphocytes, preferably induced pluripotent stem (IPS) cells,
inducing cell differentiation into lymphocytes within the cell microcompartment, so as to obtain at least lymphocytes.

### EXAMPLES

### Example 1: Method According to the Invention (T Lymphocytes)

### Reagents Used:

Solution 1: Culture medium used: TexMACS (Miltenyi Biotec) supplemented with 1000 units/mL recombinant IL-2 (Miltenyi Biotec)
Solution 2: CD3/CD28 polyantigen activation reagent (Transact™ Miltenyi Biotec)
Solution 3: Capsule rinsing medium: DMEM/F-12+ Glutamax
Solution 4: Solution for dissolving alginate capsules: non-enzymatic cell detachment buffer (RelesR™ Stem Cell Technology).

### Step 1: Cell Thawing (D0)

Ampoules of T lymphocytes sorted from healthy donor peripheral blood were thawed according to the supplier's recommendations (Hu PB Pan-T, Stem Cell Technologies, #70024).

### Step 2: T-Cell Activation (D0)

T lymphocytes were counted on the NucleoCounter NC2000 (Standard 1 cassette protocol) and cultured in solution 1 at a density of 1 million cells per mL in a culture flask.

Solution 2 was added 1:100 to the cell suspension thus prepared.

The treated cells were cultured in an incubator at 37° C. and 5% CO2 for 3 days.

### Step 3: Change of Medium and Encapsulation (D3):

To remove the Transcart™ from the medium, cells were centrifuged at 300 g for 10 min and the supernatant was sucked out.

The cells were rinsed once in solution 1.

A portion of the cell pellet was taken up in solution 1 at a cell density of 5 million cells per mL for encapsulation.

The rest of the cell pellet was taken up in solution 1 at a cell density of 5 million cells per mL (control).

### Step 4: Encapsulation (D3)

The encapsulation device is prepared as described in Alessandri et al., 2016 (“A 3D printed microfluidic device for production of functionalized hydrogel microcapsules for culture and differentiation of human Neuronal Stem Cells (hNSC)”, Lab on a Chip, 2016, vol. 16, no. 9, pp. 1593-1604).

The three required solutions are loaded onto three syringe pumps: i) alginate solution (PRONOVA® SLG100 at 2% by weight in distilled water), ii) intermediate solution (sorbitol at 300 mM), iii) cell solution (prepared in the previous step). The three solutions are coinjected concentrically using a microfluidic injector to form a jet that splits into drops with the alginate solution as the outer layer and the cell solution as the core. These drops are collected in a calcium bath (at 100 mM), which stiffens the alginate solution to form the shell.

The invention especially promotes a high expansion rate, which consequently reduces the culture time and the number of divisions to obtain a very large number of functional lymphocytes and therefore limits new mutageneses.

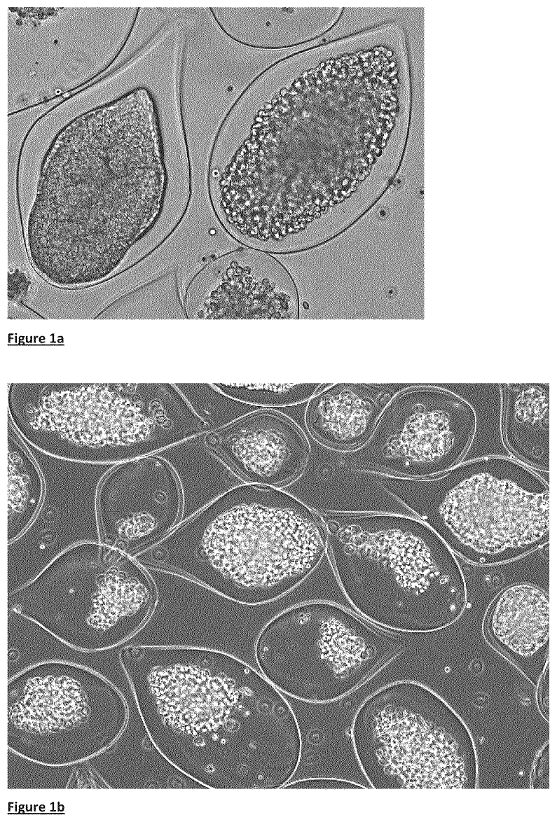

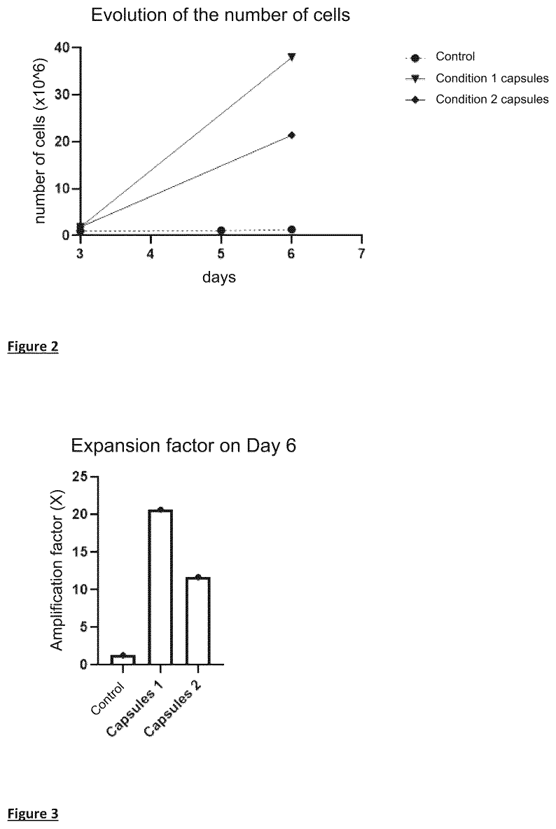

A phase contrast microscopy image at ×20 magnification of an assembly after 3 days of capsule culture is shown in FIG. 1a. A phase contrast microscopy image at ×10 magnification after 3 days of capsule culture is shown in FIG. 1b.

Step 5: Treatment after Encapsulation (D3)

The capsules are collected using a 40 μm cell sieve, then after rinsing (1×) with solution 3 they are stored in a 75 cm² flask with solution 1 at a cell density of 100,000 cells per mL (condition 1) or 200,000 cells per mL (condition 2).

The flask is kept in an incubator at 37° C. and 5% CO2 for 3 days.

Unencapsulated cells as prepared in step 3 are grown in parallel under the same atmospheric conditions for 3 days.

### Step 6: Capsule Dissolution and Counting (D6)

(a): Arm 1: capsule
The capsule suspension was collected and left to sediment for 5 minutes in a 50 mL Falcon tube.
The supernatant was carefully removed and discarded.
The capsules were then treated with solution 4 for 5 min at 37° C.
The cells released from capsules were centrifuged at 300 g for 10 minutes to remove solution 4.
Lastly, the cells were counted with NC2000 (standard protocol 1 cassette)
(b): Arm 2: suspension control
The cell suspension was collected and centrifuged at 300 g for 10 min.
The supernatant was sucked out.
Next, the cells were counted with NC2000 (standard protocol 1 cassette).

The results of T lymphocyte encapsulation using the protocol described show better cell amplification in capsules than in conventional suspension culture. In this way, we obtain a 20-fold amplification factor in 3 days of capsule culture, compared with a factor of 2 for conventional culture.

### Example 2: Measurement of Cytokine Secretion from Encapsulated and Non-Encapsulated T Lymphocytes

The detection kit used is MACSPlex Cytotoxic T/NK Cell Kit (Miltenyi Biotec, 130-125-800)

The various reagents and solutions were prepared according to the supplier's recommendations in the MACSPlex Filter Plate.

Culture supernatants from both encapsulated and non-encapsulated cells were taken at day 6 after, undiluted, and incubated in duplicate and incubated in the plate prepared in step 2 of example 1;

Incubation was carried out in accordance with the supplier's recommendations;

Data acquisition was performed on a MACSQuant 10 cytometer (Miltenyi Biotec) using a dedicated Express Mode (Miltenyi Biotec).

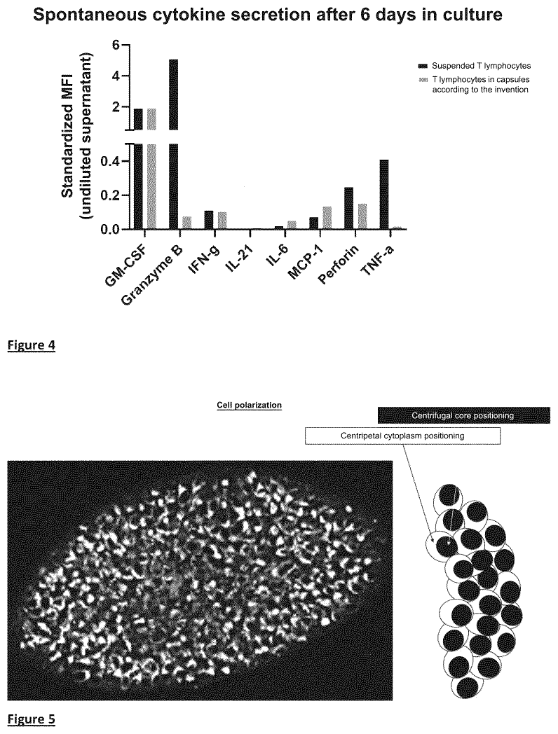

Results are expressed as mean fluorescence per million cells (MFI/e6 cells) in Table 1 and FIG. 4 (results normalized to GM-CSF and IFNg secretion).

TABLE 1

| Undiluted | | | | | | | | |
|---|---|---|---|---|---|---|---|---|
| supernatant | GM-CSF | Granzyme B | IFNg | IL-21 | IL-6 | MCP-1 | Perforin | TNF-α |
| Suspended T | 499.3 | 1339.4 | 28.9 | 1.1 | 4.6 | 18.9 | 65.4 | 108.5 |
| lymphocytes | | | | | | | | |
| Encapsulated T | 22.2 | 0.9 | 1.2 | 0.1 | 0.6 | 1.6 | 1.8 | 0.2 |
| lymphocytes | | | | | | | | |
| (invention) | | | | | | | | |
| Suspended T | 1.890571753 | 5.071563802 | 0.109428247 | 0.004165089 | 0.017417645 | 0.071563802 | 0.247633472 | 0.410829231 |
| lymphocytes | | | | | | | | |
| normalized | | | | | | | | |
| to GMCSF and | | | | | | | | |
| IFNg secretion | | | | | | | | |
| Encapsulated T | 1.897435897 | 0.076923077 | 0.102564103 | 0.008547009 | 0.051282051 | 0.136752137 | 0.153846154 | 0.017094017 |
| lymphocytes | | | | | | | | |
| normalized to | | | | | | | | |
| GMCSF and | | | | | | | | |
| IFNg secretion | | | | | | | | |
| Invention/ | 1.003630724 | 0.015167526 | 0.937272647 | 2.052059052 | 2.94425864 | 1.910912133 | 0.621265585 | 0.041608571 |
| suspension ratio | | | | | | | | |

In particular, these results confirm that lymphocytes encapsulated in the microcompartment according to the invention have a lower concentration of extracellular granzyme B, perforin and TNF alpha than lymphocytes in suspension.

### Example 3: Polarization of a Microcompartment According to the Invention

The microcompartments according to the invention comprising T lymphocytes were fixed in PFA 4% diluted in PBS with calcium and stored at 4° C.

### Staining Protocol:

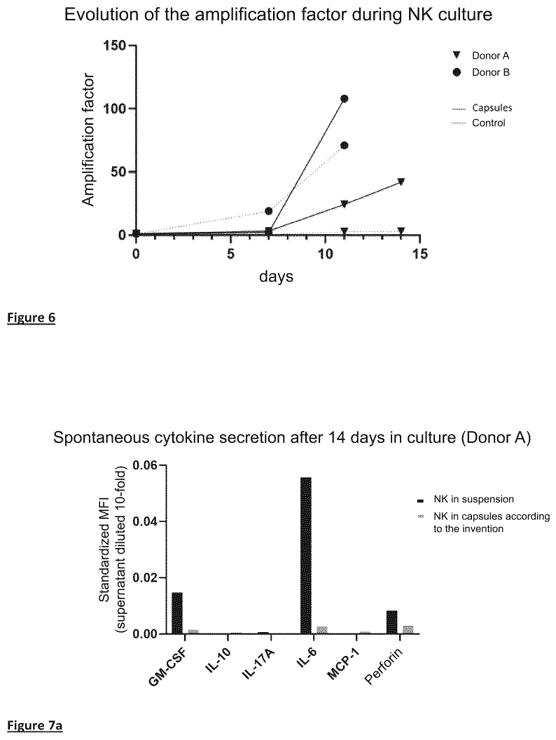

Phalloidin and Lipilight (Membright) staining were carried out at the dilutions recommended by the suppliers for 72 h under agitation, at room temperature and protected from light. Image acquisition (FIG. 5) was carried out using confocal microscopy.

A microcompartment according to the invention can be seen comprising lymphocytes with eccentric nuclei. As a result, the lymphocytes are polarized within the microcompartment and display a functional phenotype.

### Example 4: Method According to the Invention (NK Lymphocytes)

### Reagents Used:

Solution 1: Basal culture medium for NK expansion (ExCellerate Human NK Cell Expansion Media (R&D Systems)) supplemented with 27 ng/ml recombinant IL-2, 10 ng/ml recombinant IL-12, 10 ng/ml recombinant IL-18 and 10 ng/ml recombinant IL-21.
Solution 2: Solution 1 supplemented with CD2/NKp46 polyantigenic activation beads
(e.g. Cloudz Human NK Cell Expansion Kit (R&D Systems))
Solution 3: Capsule rinsing medium: DMEM/F-12+Glutamax
Solution 4: Solution for dissolving alginate capsules: 2.5 mM EDTA solution

### Step 1: Cell Thawing (D0)

NK ampoules sorted from peripheral blood of healthy donors (donor A and donor B) were thawed according to the supplier's recommendations (Hu PB NK, Stem Cell Technologies, #70036).

NKs were counted on the NucleoCounter NC2000 (Standard 1 cassette protocol).

Cells were centrifuged at 300 g for 10 min and the supernatant was sucked out.

A portion of the cell pellet was taken up in solution 2 at a cell density of 15 million cells per mL for encapsulation.

The other part of the cell pellet was taken up in solution 2 at a cell density of between 0.2 and 1 million cells per mL (control).

### Step 2: Encapsulation (D0)

The encapsulation device is prepared as described in Alessandri et al., 2016 (“A 3D printed microfluidic device for production of functionalized hydrogel microcapsules for culture and differentiation of human Neuronal Stem Cells (hNSC)”, Lab on a Chip, 2016, vol. 16, no. 9, pp. 1593-1604).

The three required solutions are loaded onto three syringe pumps: i) alginate solution (PRONOVA® SLG100 at 2% by weight in distilled water), ii) intermediate solution (sorbitol at 300 mM), iii) cell solution (prepared in the previous step). The three solutions are coinjected concentrically using a microfluidic injector to form a jet that splits into drops with the alginate solution as the outer layer and the cell solution as the core. These drops are collected in a calcium bath (at 100 mM), which stiffens the alginate solution to form the shell.

The invention especially promotes a high expansion rate, which consequently reduces the culture time and the number of divisions to obtain a very large number of functional lymphocytes and therefore limits new mutageneses.

Step 3: Treatment after Encapsulation (D0)

Capsules are collected using a 40 μm cell sieve, then after rinsing (1×) with solution 3 they are stored in a 75 cm² flask (Donor A) or in a 30 mL minibioreactor (Donor B) with solution 1 at a cell density of between 200,000 and 1,000,000 cells per mL.

The 30 mL flask or mini-bioreactor is incubated at 37° C. and 5% CO2 for 11 to 14 days. The mini-bioreactor capsules are agitated at 100 RPM. The culture medium is renewed according to the supplier's recommendations.

Unencapsulated cells as prepared in step 3 are grown in parallel under the same atmospheric conditions for 11 to 14 days.

### Step 6: Capsule Dissolution and Counting (D11 or D14, Depending on Capsule Confluence)

(a): Arm 1: capsule
The capsule suspension was collected and left to sediment for 5 minutes in a 50 mL Falcon tube.
The supernatant was carefully removed and discarded.
The capsules were then treated with solution 4 for 5 min at 37° C.
The cells released from capsules were centrifuged at 300 g for 10 minutes to remove solution 4.
The activation beads are discarded in accordance with the supplier's recommendations.
Lastly, the cells were counted with NC2000 (standard protocol 1 cassette)
(b): Arm 2: suspension control
The cell suspension was collected and centrifuged at 300 g for 10 min.
The supernatant was sucked out.
The activation beads are discarded in accordance with the supplier's recommendations.
Next, the cells were counted with NC2000 (standard protocol 1 cassette).

The results of NK encapsulation according to the protocol describe better cell amplification in capsules compared with conventional suspension culture, as shown in FIG. 6. In this way, for donor A, we obtain a 40-fold amplification factor in 14 days of encapsulation culture, compared with a factor of 3 for conventional culture. For donor B, we obtain a 110-fold amplification factor in 11 days of encapsulation culture, compared with a factor of 70 for conventional culture.

FIG. 10 shows that the viability of NKs obtained according to the invention is better than that of NKs cultured in 2D. For donor A, viability at D14 was 74% for encapsulation culture versus 64% for suspension culture. For donor B, viability at D11 was 88% in encapsulation culture versus 62% in suspension.

### Example 5: Measurement of Cytokine Secretion from Encapsulated and Non-Encapsulated NK Lymphocytes

The detection kit used is MACSPlex Cytotoxic T/NK Cell Kit (Miltenyi Biotec, 130-125-800)

The various reagents and solutions were prepared according to the supplier's recommendations in the MACSPlex Filter Plate.

Culture supernatants from encapsulated or non-encapsulated cells were collected 14 days later, diluted 10-fold or 100-fold, and incubated in duplicate in the plate prepared in step 2 of example 4;

Incubation was carried out in accordance with the supplier's recommendations; Data acquisition was performed on a MACSQuant 10 cytometer (Miltenyi Biotec) using a dedicated Express Mode (Miltenyi Biotec).

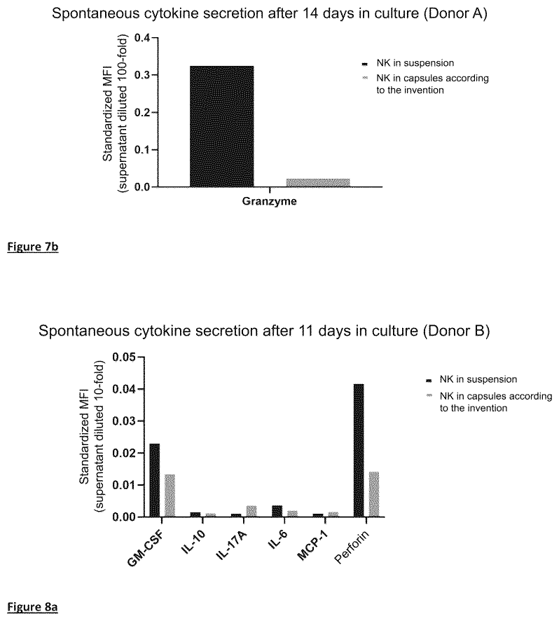

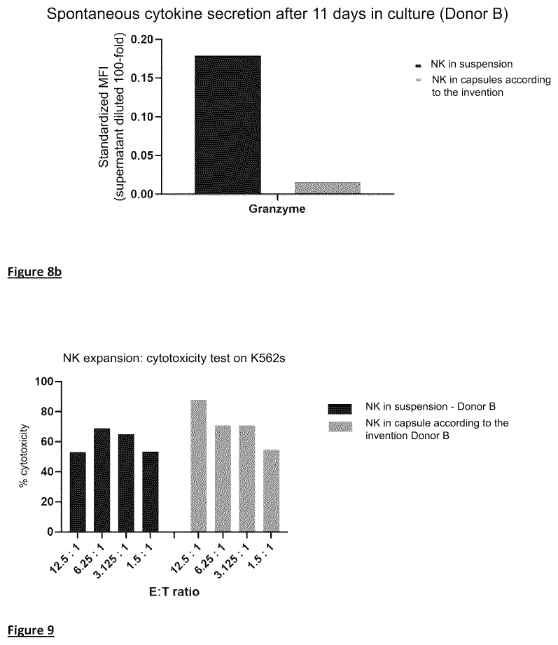

Results are expressed as mean fluorescence per million cells (MFI/e6 cells) in Tables 2 and 3 and in FIGS. 7a, 7b (Donor A) and 8a, 8b (Donor B).

TABLE 2

| 10-fold diluted supernatant | GM-CSF | IL-10 | IL-17A | IL-6 | MCP-1 | Perforin |
|---|---|---|---|---|---|---|
| NK, donor A in suspension | 168.3 | 1.2 | 8 | 635 | 3.8 | 94.7 |
| NK, A donor in capsule (invention) | 14.8 | 5.5 | 0.8 | 25 | 7.7 | 27 |
| NK, B donor in suspension | 60.9 | 3.9 | 2.7 | 9.6 | 2.8 | 110.3 |
| NK, B donor in capsule (invention) | 3.3 | 0.3 | 0.9 | 0.5 | 0.4 | 3.5 |
| NK, donor A in suspension normalized to | 0.014763158 | 0.000105263 | 0.000701754 | 0.055701754 | 0.000333333 | 0.008307018 |
| IFNg secretion | | | | | | |
| NK, donor A in capsule normalized to | 0.001679337 | 0.000624078 | 9.0775E−05 | 0.002836718 | 0.000873709 | 0.003063656 |
| IFNg secretion | | | | | | |
| NK, donor B in suspension normalized to | 0.022989807 | 0.001472254 | 0.001019253 | 0.003624009 | 0.001057003 | 0.041638354 |
| IFNg secretion | | | | | | |
| NK, donor B in capsule normalized to | 0.013414634 | 0.001219512 | 0.003658537 | 0.00203252 | 0.001626016 | 0.014227642 |
| IFNg secretion | | | | | | |
| Invention/donor A suspension ratio | 0.113751906 | 5.928741632 | 0.129354363 | 0.050926915 | 2.621127879 | 0.368803358 |
| Invention/donor B suspension ratio | 0.583503544 | 0.828330206 | 3.589430894 | 0.560848577 | 1.538327526 | 0.341695597 |

TABLE 3

| 100-fold diluted supernatant | Granzyme | IFNg |
|---|---|---|
| NK, donor A in suspension | 3700 | 11400 |
| NK, A donor in capsule | 208 | 8813 |
| (invention) | | |
| NK, B donor in suspension | 474 | 2649 |
| NK, B donor in capsule | 4 | 246 |
| (invention) | | |
| NK, donor A in capsule | 0.324561404 | 1 |
| normalized to IFNg | | |
| secretion | | |
| NK, donor A in suspension | 0.023601498 | 1 |
| normalized to IFNg | | |
| secretion | | |
| NK, donor B in suspension | 0.178935447 | 1 |
| normalized to IFNg | | |
| secretion | | |
| NK, donor B in capsule | 0.016260163 | 1 |
| normalized to IFNg | | |
| secretion | | |
| Invention/donor A | 0.072718128 | 1 |
| suspension ratio | | |
| Invention/donor B | 0.090871668 | 1 |
| suspension ratio | | |

In particular, these results confirm that NK lymphocytes encapsulated in the microcompartment according to the invention have a lower concentration of granzyme B, perforin, GM-CSF and IL-6 than NK lymphocytes in suspension.

### Example 6: Measuring the Functionality of Encapsulated and Non-Encapsulated NK Lymphocytes

Measurement was performed by flow cytometry on K562s according to the following protocol.

After decapsulation, stain the NKs (B donor NKs obtained according to the procedures in Example 4, capsule arm and control arm in suspension) with the PKH-26 probe, then incubate the NKs for 4 h at 37° C. 5% CO2 with K562 at different E:T ratios (effector to target): 12.5:1/6.25:1/3.125:1/1.5:1. Annexin V and 7-AAD staining. The cytometry data analysis strategy was performed as follows: separation of NK and K562 populations using the PKH-26 probe (K562=negative population):
live K562s are negative for 7aad−/AnnexinV−
Early apoptotic K562s are 7AAD−/AnnexinV+
Late apoptotic K562s are 7AAD+/AnnexinV+
Necrotic or dead K562s are 7AAD+/AnnexinV−.

The percentage of cytotoxicity was calculated as follows:

% Cytotoxicity=(early apoptosis+late apoptosis+necrosis)/total K562

The results obtained are shown in FIG. 9. The cytotoxicity of NK obtained according to the invention is similar to that of NK obtained in 2D.

### Example 7 Method According to the Invention (NK Lymphocytes)

### Reagents Used:

Solution 1: Basal culture medium for NK expansion (ExCellerate Human NK Cell Expansion Media (R&D Systems)) supplemented with 27 ng/ml recombinant IL-2, 10 ng/ml recombinant IL-12, 10 ng/ml recombinant IL-18 and 10 ng/mL recombinant IL-21.
Solution 2: Capsule rinsing medium: DMEM/F-12+Glutamax
Solution 3: Solution for dissolving alginate capsules: 2.5 mM EDTA solution

### Step 1: Cell Thawing (D0)

NK ampoules sorted from peripheral blood of healthy donors (donor B and donor C) were thawed according to the supplier's recommendations (Hu PB NK, Stem Cell Technologies, #70036).

NKs were counted on the NucleoCounter NC2000 (Standard 1 cassette protocol).

Cells were centrifuged at 300 g for 10 min and the supernatant was sucked out.

A portion of the cell pellet was taken up in solution 1 at a cell density of 5 to 15 million cells per mL for encapsulation.

The rest of the cell pellet was taken up in solution 1 at a cell density of 0.2 to 1 million cells per mL (control).

### Step 2: Encapsulation (D0)

The encapsulation device is prepared as described in Alessandri et al., 2016 (“A 3D printed microfluidic device for production of functionalized hydrogel microcapsules for culture and differentiation of human Neuronal Stem Cells (hNSC)”, Lab on a Chip, 2016, vol. 16, no. 9, pp. 1593-1604).

The three required solutions are loaded onto three syringe pumps: i) alginate solution (PRONOVA® SLG100 at 2% by weight in distilled water), ii) intermediate solution (sorbitol at 300 mM), iii) cell solution (prepared in the previous step). The three solutions are coinjected concentrically using a microfluidic injector to form a jet that splits into drops with the alginate solution as the outer layer and the cell solution as the core. These drops are collected in a calcium bath (at 100 mM), which stiffens the alginate solution to form the shell.

The invention especially promotes a high expansion rate, which consequently reduces the culture time and the number of divisions to obtain a very large number of functional lymphocytes and therefore limits new mutageneses.

Step 3: Treatment after Encapsulation (D0)

Capsules are collected using a 40 μm cell sieve, then after rinsing (1×) with solution 2 they are stored in a 30 mL mini-bioreactor with solution 1 at a cell density of 200,000 cells per mL.

The 30 mL mini-bioreactors are incubated at 37° C. and 5% CO2 for 11 days. The mini-bioreactor capsules are agitated at 100 RPM. The culture medium is renewed according to the supplier's recommendations.

Unencapsulated cells as prepared in step 14 are grown in parallel under the same atmospheric conditions for 11 days.

### Step 6: Capsule Dissolution and Counting (D11)

(a): Arm 1: capsule
The capsule suspension was collected and left to sediment for 5 minutes in a 50 mL Falcon tube.
The supernatant was carefully removed and discarded.
The capsules were then treated with solution 3 for 5 min at 37° C.
The cells released from capsules were centrifuged at 300 g for 10 minutes to remove solution 3.
Lastly, the cells were counted with NC2000 (standard protocol 1 cassette)
(b): Arm 2: suspension control
The cell suspension was collected and centrifuged at 300 g for 10 min.
The supernatant was sucked out.
Next, the cells were counted with (standard protocol 1 cassette).

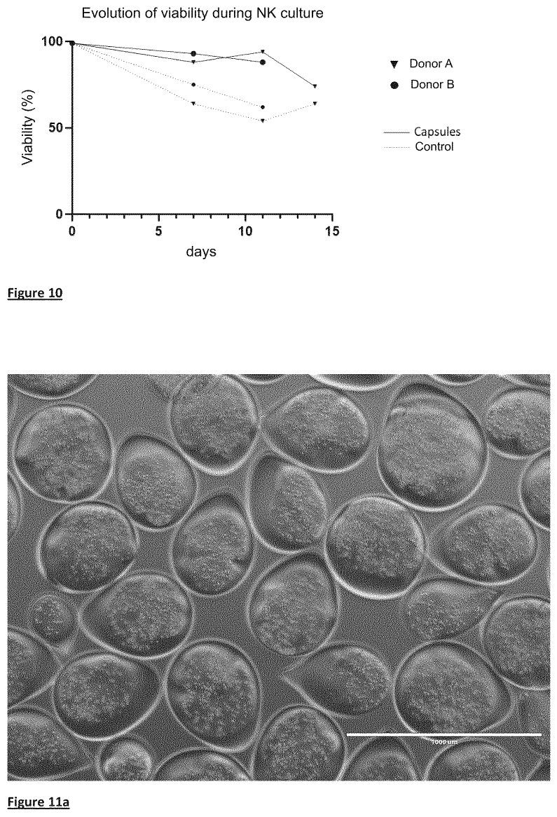

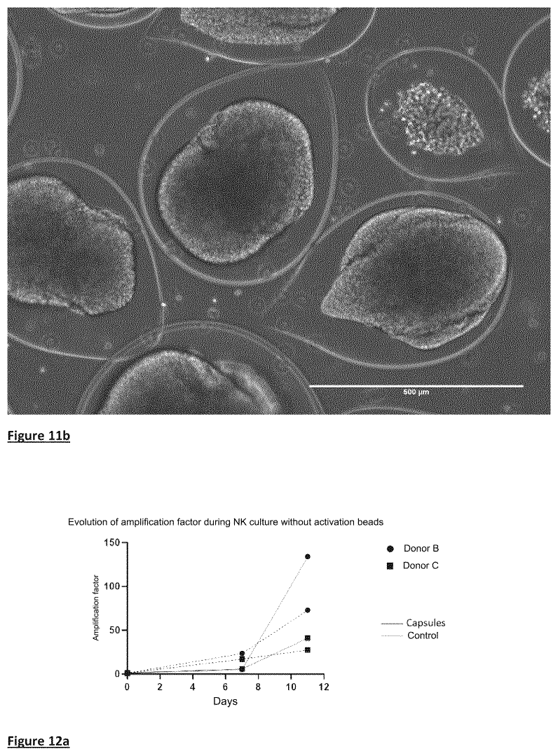

A phase contrast microscopy image at ×4 magnification of an assembly on the day of encapsulation is shown in FIG. 11a. A phase contrast microscopy image at ×10 magnification after 11 days of capsule culture is shown in FIG. 11b.

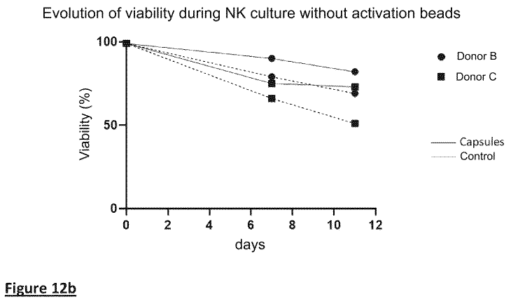

The results of NK encapsulation according to the protocol describe better cell amplification in capsules compared with conventional suspension culture, as shown in FIG. 12a In this way, for donor B, the amplification factor is 135-fold in 11 days of capsule culture, compared with a factor of 72 for conventional culture. For donor C, the amplification factor is 41-fold in 11 days of capsule culture, compared with a factor of 28 for conventional culture.

FIG. 12b shows that the viability of NKs obtained according to the invention is better than that of NKs cultured in 2D. For donor B, viability at D11 is 82% for encapsulation culture versus 69% for suspension culture. For donor C, viability at D11 is 73% in encapsulation culture versus 51% in suspension.

1. A closed three-dimensional cellular microcompartment of ovoid, cylindrical, spheroid or spherical shape or of substantially ovoid, cylindrical, spheroid or spherical shape, the smallest dimension of which is between 200 and 400 μm, comprising an outer hydrogel layer that delimits an internal part, said internal part comprising, in a medium, between 500 and 5000 lymphocytes forming a grouped three-dimensional culture and having a TNF alpha content of less than 100 ng/ml of the medium.

2. The microcompartment according to claim 1, characterized in that the internal part comprises lymphocytes forming a grouped three-dimensional culture without lumens.

3. The microcompartment according to claim 1, characterized in that it has been obtained after encapsulation of lymphocytes without addition of extracellular matrix or without addition of extracellular matrix elements.

4. The microcompartment according to claim 1, characterized in that it has been obtained after encapsulation of previously activated lymphocytes.

5. The microcompartment according to claim 1, characterized in that the thickness of the outer layer is variable and between 20 and 60 μm.

6. The microcompartment according to claim 1, characterized in that the lymphocytes are polarized.

7. The microcompartment according to claim 1, characterized in that the number of lymphocytes forming a grouped three-dimensional culture is between 500 and 5000.

8. The microcompartment according to claim 1, characterized in that the number of lymphocytes forming a grouped three-dimensional culture is between 1000 and 3000.

9. The microcompartment according to claim 1, characterized in that the lymphocytes are T lymphocytes or NK lymphocytes.

10. The microcompartment according to claim 1, characterized in that the lymphocytes are lymphocytes expressing a chimeric antigen receptor.

11. The microcompartment according to claim 1, characterized in that the cells are T lymphocytes and in that it has:
a perforin content of less than 0.5 μg/mL of medium, and/or
a granzyme B content of less than 1 μg/mL of medium.

12. The microcompartment according to claim 1, characterized in that the lymphocytes are NK lymphocytes and in that it has:
a perforin content of less than 0.5 μg/mL of medium, and/or
a granzyme B content of less than 1 μg/mL of medium, and/or
a perforin IL6 content of less than 0.5 μg/mL of medium, and/or
a GM-CSF content below 0.5 μg/mL of medium.

13. The microcompartment according to claim 1, characterized in that the external layer comprises alginate.

14. The microcompartment according to claim 1, characterized in that the internal part comprises a solution comprising IL2 or the combination of IL7 and IL 15 or IL 12, IL18, IL21 or the combination of at least two cytokines chosen from IL2, IL12, IL 18 and IL21.

15. The microcompartment according to claim 1, characterized in that it comprises T lymphocytes and in that the proportion of naive and/or Tscm T lymphocyte remains greater than 20%.

16. An assembly of microcompartments comprising at least two three-dimensional cellular microcompartments, characterized in that at least one microcompartment is a microcompartment according to claim 1.

17. The assembly of microcompartments according to claim 16, characterized in that the microcompartments are arranged in a culture medium in a bioreactor.

18. The microcompartment according to claim 1, for use thereof as a drug.

19. The microcompartment according to claim 1, for use in the prevention or treatment of autoimmune diseases, immunodeficiency syndromes, cancers, viral diseases or inflammatory diseases.

20. A method for preparing a microcompartment according to claim 1 or an assembly of at least two of these cellular microcompartments, comprising the following steps:
(a) incubating lymphocytes in a culture medium comprising cytokines,
(b) encapsulating lymphocytes in their culture medium in a hydrogel layer, preferably without adding extracellular matrix, to form a closed three-dimensional microcompartment of ovoid, cylindrical, spheroid or spherical shape or substantially ovoid, cylindrical, spheroid or spherical shape, the smallest dimension of which is between 200 and 400 μm, so that each microcompartment comprises at least 5 lymphocytes in its internal part,
(c) culturing the microcompartments obtained in step (b) in a culture medium comprising at least cytokines, for 2 to 6 days.

21. The method for preparing a microcompartment according to claim 20, characterized in that it comprises a step prior to the incubation of lymphocytes, which consists in activating said lymphocytes.

22. The method for preparing a microcompartment according to claim 20, characterized in that the culture media comprise 100 to 1000 units/mL of IL2 or the combination of 300 to 600 units/mL of IL7 and 50 to 100 units/mL of IL 15.

23. The method for preparing a microcompartment according to claim 20, characterized in that in step (a) between 0.5 and 2 million lymphocytes are incubated per mL of culture medium.

24. The method according to claim 20, characterized in that step b) is carried out by co-injection of two solutions:
a hydrogel solution,
the solution from step a) comprising lymphocytes and culture medium, concentrically via a microfluidic injector which makes it possible to form a jet at the outlet of the injector consisting of the mixture of the two solutions, said jet breaking up into droplets, said droplets being collected in a calcium bath which stiffens the hydrogel solution to form the external layer of each microcompartment, the internal part of each droplet consisting of the solution coming from step (a) comprising lymphocytes and culture medium.

25. The method according to claim 24, characterized in that the microfluidic injector has a final opening diameter of between 150 and 300 μm.

26. The method according to claim 20, characterized in that the flow rate of each of the two solutions is between 45 and 150 mL/h.

27. The method according to claim 20, characterized in that all of the cells initially encapsulated in step (b) represents a volume less than 50% of the volume of the microcompartment in which they are encapsulated.

28. The method according to claim 20, characterized in that steps (a), and/or (c) are carried out under permanent or sequential stirring.

29. The method according to claim 20, characterized in that the method comprises at least one re-encapsulation of the cells after step (c).

30. The method according to claim 29, characterized in that each re-encapsulation consists in removing the external hydrogel layer, preferentially in resuspending, in a partially or totally dissociated manner, the lymphocytes which were partially or totally dissolved in the microcompartments, and in re-implementing the steps of the method.

31. The method for preparing a microcompartment according to claim 20, characterized in that it comprises a step after step (c) which consists in freezing the microcompartments.

32. The method for preparing a microcompartment according to claim 20, characterized in that step (c) is implemented in a bioreactor.
